(19) United States
(12) Patent Application Publication
(10) Pub. No.: US 2004/0072842 A1
(43) Pub. Date: Apr. 15, 2004

(54) Difluorothioacetamides of oxazolidinones with a glycoloylpiperazine substituent

(76) Inventors: Jackson Boling Hester JR., Galesburg, MI (US); Wade J. Adams, Kalamazoo, MI (US); Jeffrey Charles Stevens, Kalamazoo, MI (US); Mikhail Fedor Gordeev, Castro Valley, CA (US); Upinder Singh, Freemont, CA (US); Carole Scott, Newark, CA (US)

Correspondence Address: PHARMACIA & UPJOHN, 301 HENRIETTA ST, 0228-32-LAW, KALAMAZOO, MI, 49007, US

(21) Appl. No.: 10/462,332

(22) Filed: Jun. 16, 2003

Related U.S. Application Data
(60) Provisional application No. 60/392,716, filed on Jun. 28, 2002.

(51) Int. Cl. C07D 413/02; A61K 31/496; A61K 31/454

(52) U.S. Cl. 514/254.02; 514/326; 544/369; 546/209

(57) ABSTRACT

The present invention describes difluororthioacetamide oxazolidinones with a glycoloylpiperazine substituent as novel antibacterial agents, and antimicrobial combination therapies for combating infective diseases caused by gram-positive and gram-negative bacteria.

### CROSS REFERENCE

This application claims the benefit of the following provisional application: U.S. Serial No. 60/392,716, filed Jun. 28, 2002, under 35 USC 119(e)(i), which is incorporated herein by reference in its entirety.

### FILED OF THE INVENTION

The present invention describes difluororthioacetamide oxazolidinones with a glycoloylpiperazine substituent as novel antibacterial agents, and antimicrobial combination therapies for combating infective diseases caused by gram-positive and gram-negative bacteria.

### BACKGROUND OF THE INVENTION

The thioamide oxazolidinone antibacterial agents are a novel synthetic class of antimicrobials with broad activity against a number of human and veterinary pathogens, including gram-positive aerobic bacteria such as multiply-resistant staphylococci and streptococci, gram-negative aerobic bacteria such as H. influenzae and M. catarrahlis, as well as anaerobic organisms such as bacteroides and clostridia species, and acid-fast organisms such as Mycobacterium tuberculosis and Mycobacterium avium.

As a chemical compound class, thioamide oxazolidinones generally are rapidly metabolized. It is known in the pharmaceutical filed that compounds with minimum metabolism are preferred to rapidly metabolized compounds for several reasons. It is easier to maintain therapeutic blood levels of slowly metabolized compounds (active ingredients) since they typically have lower clearance than rapidly metabolized compounds. Blood levels in humans are more predictable for slowly metabolized compounds since there is less effect from normal human variability in enzyme levels and activity. Metabolized compounds may also generate toxic metabolites, whereas non-metabolized compounds do not.

Accordingly, there is a demand to discover thioamide oxazolidinone antibacterial agents that possess minimum metabolism. Difluorothioacetamide oxazolidinones of the present invention have potent activity against gram-positive human and veterinary pathogens. In particular, it is unexpectedly discovered that these compounds have good stability in vivo and a very low metabolism rate.

### INFORMATION DISCLOSURE

U.S. Pat. No. 6,342,513 discloses Oxazolidinone antibacterial agents having a thiocarbonyl functionality.

U.S. Pat. No. 6,281,210 discloses Benzoic acid esters of oxazolidinones having a hydroxyacetylpiperazine substituent.

U.S. Pat. No. 6,166,056 discloses phenyloxazolidinones having a C—C bond to 4-6 membered heterocyclic rings.

International publication WO 01/58885 discloses oxazolidinone thioamides with piperazine amide substituents.

### SUMMARY OF THE INVENTION

The present invention provides a novel oxazolidinone compound of formula I

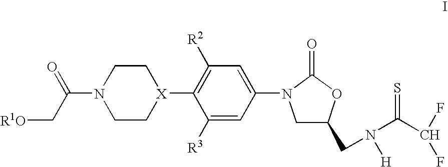

wherein X is N or CH;

R² and R³ are independently H or F;

R¹ is H, —CH₂phenyl, or —C(═O)C₁₋₄alkyl.

The present invention further provides a method for treating gram-positive bacterial infections which comprises administration to a mammal being treated a pharmaceutically effective amount of the compound of formula I, either individually, or in combination with other gram-positive antibiotics.

The present invention further provides a method for treating gram-positive and gram-negative bacterial infections which comprises administration to a mammal being treated a pharmaceutically effective amount of the compound of formula I in combination with at least one other gram-negative antibiotic.

The present invention further provides compositions for treating gram-positive bacterial infections wherein the compositions comprise a pharmaceutically effective amount of the compound of formula I and at least one other gram-positive antibiotic.

The present invention further provides compositions for treating gram-positive and gram-negative bacterial infections wherein the compositions comprise a pharmaceutically effective amount of the compound of formula I and at least one other gram-negative antibiotic.

The present invention further provides a compound of formula Ia as a intermediate useful for preparing a compound of formula I.

The present invention further provides methods of preparation of the compounds of formula I of the present invention.

The present invention further provides a use of the compound of formula I to prepare a medicament, for treating gram-positive and/or gram-negative bacterial infections.

### DETAILED DESCRIPTION OF THE INVENTION

Definitions

The term “antibiotic” refers to an antibacterial agent other than the compound of the present invention.

Specifically, they refer to Amikacin, Gentamicin, Spectinomycin, Tobramycin, Imipenem, Meropenem, Cefadroxil, Cefazolin, Cephalexin, Cefaclor, Cefotetan, Cefoxitin, Cefprozil, Cefuroxime, Loracarbef, Cefdinir, Cefixime, Cefoperazone, Cefotaxime, Cefpodoxime, Ceftazidime, Ceftibuten, Ceftozoxime, Ceftriaxone, Cefepime, Azithromycin, Clarithromycin, Dirithromycin, Penicillin G, Cloxacillin, Dicloxacillin, Nafcillin, Oxacillin, Amoxicillin, Amoxicillin, Ampicillin, Mezlocillin, Piperacillin, Nalidixic Acid, Ciprofloxacin, Enoxacin, Lomefloxacin, Norfloxacin, Ofloxacin, Levofloxacin, Sparfloxacin, Alatrofloxacin, Gatifloxacin, Moxifloxacin, Trimethoprim, Sulfisoxazole, Sulfamethoxazole, Doxycycline, Minocycline, Tetracycline, Aztreonam, Chloramphenicol, Clindamycin, Quinupristin, Fosfomycin, Metronidazole, Nitrofurantoin, Rifampin, Trimethoprim, and Vancomycin. All of them are known. They can be either obtained commercially or be prepared according to the references cited in PHYSICIANS' DESK REFERENCE, the 53^{rd} Edition (1999) and the US FDA's Orange book.

The term “gram-positive antibiotic” refers to an antibacterial agent active against gram-positive bacterial organisms.

The term “gram-negative antibiotic” refers to an antibacterial agent active against gram-negative bacterial organisms.

For the purpose of the present invention, the carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, the term “C₁₋₄alkyl refers to alkyl of one to four carbon atoms, inclusive, or methyl, ethyl, propyl, and butyl, straight and branched forms thereof.

Specifically, R² and R³ are H.

Specifically, R² and R³ are F.

Specifically, R² is H and R³ is F.

Specifically, R² is F and R³ is H.

Specifically R¹ is H.

Specifically R¹ is —CH₂phenyl.

Specifically R¹ is —C(═O)C₁₋₄alkyl.

Specifically, R¹ is —C(═O)CH₂phenyl

Specifically, X is N.

Specifically X is CH.

Examples of the present invention are:

(a) 2,2-difluoro-N-({(5S)-3-[3,5-difluoro-4-(4-glycoloyl-piperazine-1-yl)phenyl]-2-oxo-1,3-oxazolidin-5-yl}methyl)ethanethioamide,

(b) 2-{4-[4-((5S)-5-{[(2,2-difluoroethanethiolyl)-amino]methyl}-2-oxo-1,3-oxazolidin-3-yl)-2-fluorophenyl]piperazin-1-yl}-2-oxoethyl Acetate,

(c) 2,2-difluoro-N-({(5S)-3-[3-fluoro-4-(4-glycoloylpiperazin-1-yl)phenyl]-2-oxo-1,3-oxazolidin-5-yl}methyl)ethanethioamide,

(d) 2,2-difluoro-N-({(5S)-3-[4-glycoloylpiperazin-1-yl)phenyl]-2-oxo-1,3-oxazolidin-5-yl}methyl)-ethanethioamide,

(e) N-{[(5S)-3-(4-{4-[(benzyloxy)acetyl]piperazin-1-yl}phenyl)-2-oxo-1,3-oxazolidin-5-yl]methyl}-2,2-difluoroethanethioamide,

(f) 2,2-difluoro-N-({(5S)-3-[3-fluoro-4-(1-glycoloylpiperidin-4-yl)phenyl]-2-oxo-1,3-oxazolidin-5-yl}methyl)ethanethioamide, and

(g) 2,2-difluoro-N-({(5S)-3-[4-(1-glycoloylpiperidin-4-yl)phenyl]-2-oxo-1,3-oxazolidin-5-yl}methyl)ethanethioamide.

Description for the Preparations

Compounds of formula I of the present invention can be prepared as illustrated in Schemes I and II, wherein X, R¹, R² and R³ are as described previously or in claims. In Scheme I, R⁴ represents hydrogen, —C(═O)CH₂OR¹ or suitable amine protecting groups such as tert-butoxycarbonyl (Boc) and benzyloxycarbonyl (Cbz). The starting material, amines (II), can be prepared according to the procedure described in U.S. Pat. No. 6,342,523. Where R⁴ in the amine II is —C(═O)CH₂OR¹ or suitable amine protecting groups, they are allowed to react with an ester of difluoroethanethioic O-acid IV wherein R⁵ is C₁₋₄ alkyl optionally substituted by one or two phenyl groups. Suitable solvents for this reaction include methanol, chloroform, methylene chloride or mixtures thereof at temperatures of about 10° C. to about 30° C. A tertiary amine base such as triethylamine can be used to facilitate this reaction, especially if a salt of the amine II is employed. As illustrated in Example 1 the Boc protecting group can be removed with acid catalysts such as trifluoroacetic acid in methylene chloride or 4N hydrogen chloride in dioxane at temperatures of about 0° C. to about 25° C. Removal of the Cbz group can be carried out with about 20% hydrogen bromide in acetic acid at temperatures about 0° C. to about 30° C. The remaining steps which lead from the resulting compounds wherein R⁴ is hydrogen to compounds of formula I are shown in Scheme II.

An alternative method for preparing compounds of formula I is illustrated in Scheme II. Condensation of a compound of structure V, wherein R⁶ is a protecting group such as Boc or Cbz, with difluoroacetic acid provides the difluoroacetamide VI. Reagents and conditions for this condensation include the use of 1-(3-dimethylaminopropyl)-3-ethylcarbodiimide hydrochloride (EDC) with 4-(dimethylamino)pyridine (DMAP) in pyridine at temperature of about 0° C. to about 25° C. or EDC with 1-hydroxybenzotriazole hydrate (HOBT) and triethylamine in DMF at temperature of about 0° C. to about 25° C. The protecting groups R⁶ can then be removed to give compounds VII which can be converted to the thioamide VIII with Lawesson's Reagent. The reaction of VII with Lawesson's Reagent is facilitated by the use of 1,3-dimethyl-3,4,5,6-tetrahydro-2(1H)-pyrimidone (DMPU) and can be carried out in solvents such as THF or dioxane at temperatures of about 20° C. to about 100° C. Condensation of the amines VIII with activated carboxylic acid derivatives will then give compounds of formula I. The reaction of VIII with acetoxyacetyl chloride and triethylamine in methylene chloride at temperature of about 0° C. to about 25° C., for example, can be used to prepare I where R¹ is acetyl. Condensing agents such as EDC with the appropriate acids, as described above, can also be used for this reaction. Compounds where R¹ is acetyl can be hydrolyzed to the corresponding compounds where R¹ is hydrogen with aqueous potassium carbonate in methanol as illustrated in Example 3.

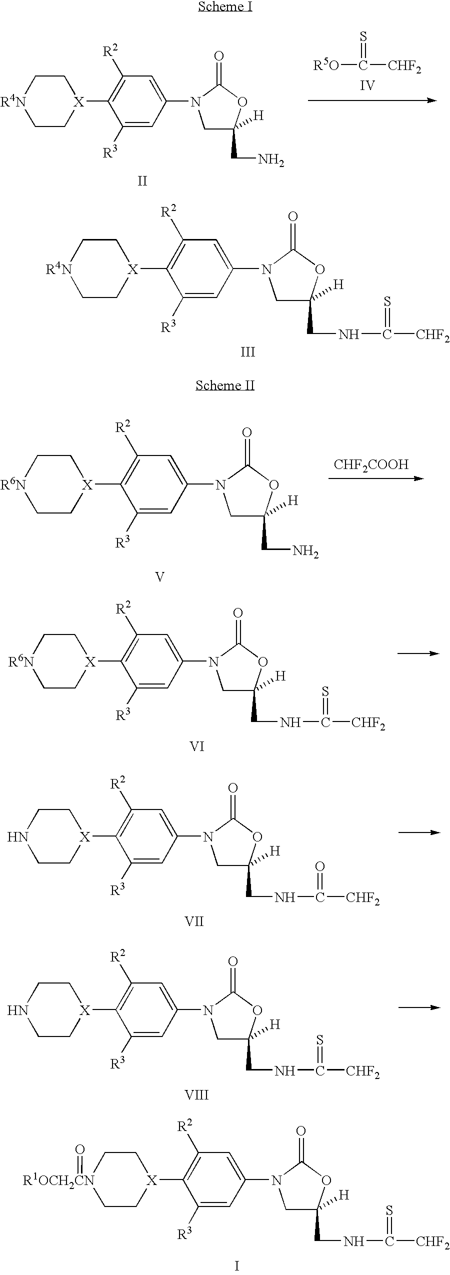

### EXAMPLES

### Example 1

### Preparation of 2,2-difluoro-N-({(5S)-3-[3,5-difluoro-4-(4-glycoloyl-piperazine-1-yl)phenyl]-2-oxo-1,3-oxazolidin-5-yl}methyl)ethanethioaamide(6)

Step 1. Preparation of tert-butyl 4-[4-((5S)-5-{[(2,2-difluoroethanethioyl)amino]methyl}-2-oxo-1,3-oxazolidin-3-yl)-2,6-difluorophenyl]piperazine-1-carboxylate (2).

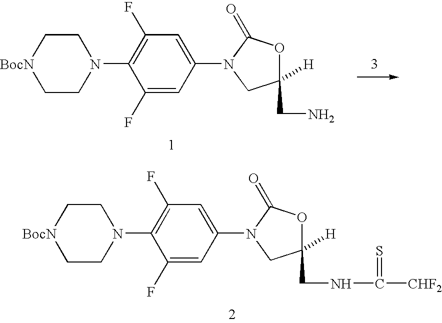

A stirred solution of 1 (prepared according to the procedure described in U.S. Pat. No. 6,342,523, example 29) (361 mg, 0.88 mmol) in 50% MeOH—CH₂Cl₂ (10 ml) is treated with O-(3,3-diphenylpropyl)difuoroethanethioate 3 (300 mg, 0.98 mmol), kept at ambient temperature (24° C.) for 30 min and concentrated. Flash chromatography of the residue on silica gel with 3% MeOH—CHCl₃ gave 546 mg of 2, a colorless oil.

Method for Preparing O-(3,3-diphenylpropyl)difuoroethanethioate 3:

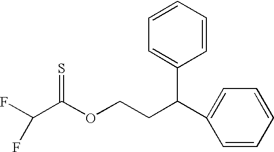

To a stirred solution of difluoroacetic acid (5.00 g, 52.1 mmol) and 3,3-diphenyl-1-propanol (11.4 ml, 57.3 mmol, 1.10 eq) in diethyl ether (100 ml) is added 4-dimethylaminopyridine (0.64 g, 5.21 mmol, 0.01 eq) followed by diisopropylcarbodiimide (6.56 g, 52.1 mmol, 1.0 eq). The mixture is stirred at room temperature overnight. The precipitate is removed by vacuum filtration and washed with ether and the filtrate concentrated in vacuo. The residue is filtered through a plug of silica gel using 5% ether/hexanes eluent and the filtrate collected and concentrated. The resulting compound (14.40 g, 46.64 mmol) in xylenes (150 ml) is added Lawesson's Reagent (24.1 g, 59.61 mmol). The reaction mixture is heated at reflux overnight and then cooled to room temperature. A precipitate formed which is removed by vacuum filtration and washed with ethyl acetate. The filtrate is passed through a plug of silica gel and eluted with 5% ether/hexanes and concentrated to give the title compound 3. ¹H NMR (400 Mhz), CDCl₃) δ 2.47, 4.07, 4.24, 5.82, 7.23.

Step 2. Preparation of N-{[(5S)-3-(3,5-difluoro-4-piperazin-1-ylphenyl)-2-oxo-1,3-oxazolidin-5-yl]methyl}-2,2-difluoroethanethioamide trifluoroacetate (4).

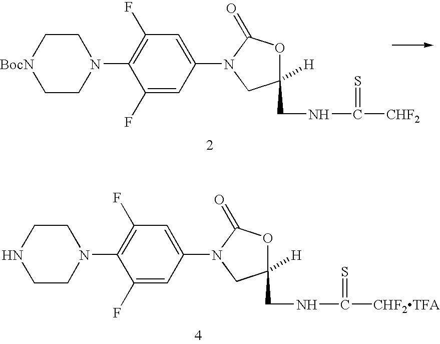

A stirred mixture of 2 (576 mg) and trifluoroacetic acid (5 ml) is kept under nitrogen, at ambient temperature (24° C.) for 30 min, diluted with methylene chloride and concentrated to give the title compound (4) as oil.

Step 3. Preparation of 2-{4-[4-((5S)-5-{[(2,2-difluoroethanethioyl)amino]methyl}-2-oxo-1,3-oxazolidin-3-yl)-2,6-difluorophenyl]piperazin-1-yl}-2-oxoethyl acetate (5).

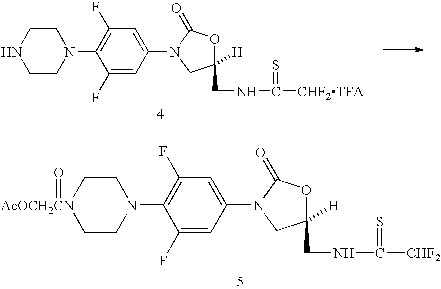

A stirred solution of 4, the product from Step 2, and triethylamine (268 μL, 1.92 mmol) in CH₂Cl₂ (10 ml) is cooled, under nitrogen, to 0° C. and treated, dropwise with acetoxyacetyl chloride (103 μL, 0.96 mmol). It is kept at 0° C. for 30 min, diluted with CH₂Cl₂ and washed with saturated NaHCO₃ and brine. The organic solution is dried (Na₂SO₄) and concentrated to give 679 mg of the title compound (5) as oil.

Step 4. Preparation of 2,2-difluoro-N-({(5S)-3-[3,5-difluoro-4-(4-glycoloyl-piperazine-1-yl)phenyl]-2-oxo-1,3-oxazolidin-5-yl}methyl)ethanethio amide(6).

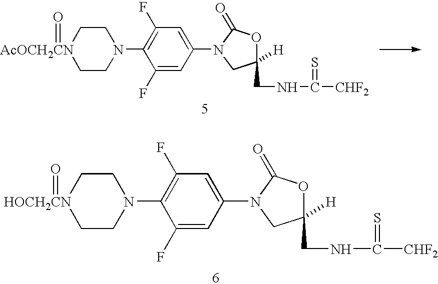

A stirred solution of 5 (679 mg) in MeOH (10 ml) is cooled to 0° C., under nitrogen and treated with 10% aqueous K₂CO₃ (1 ml). It is kept at ambient temperature (24° C.) for 30 min, treated with additional 10% K₂CO₃ (1 ml), kept for 60 min, treated with additional 10% K₂CO₃ (0.5 ml) and stirred for 30 min. The resulting solution is mixed with saturated NaHCO₃ and extracted with 5% MeOH—CH₂Cl₂. The extract is dried and concentrated. Flash chromatography of the residue on silica gel with 25-35% EtOAc-1% MeOH—CHCl₃ followed by 5% MeOH—CHCl₃ and crystallization of the product from EtOAc-heptane gave 163 mg of the title compound (6).

Physical data: mp 91-93° C. (dec).

MS (ESI+) m/z 465 (M+H⁺), 487 (M+Na⁺); MS (ESI−) m/z 463 (M−H).

HRMS calcd for C₁₈H₂₁F₄N₄O₄S (M+H⁺) 465.1219, found 465.1221.

Anal calcd for C₁₈H₂₀F₄N₄O₄S: C, 46.55; H, 4.34; N, 12.06. Found: C, 46.94; H, 4.57; N, 11.47.

### Example 2

### Preparation of 2-{4-[4-((5S)-5-{[(2,2-difluoroethanethiolyl)amino]methyl}-2-oxo-1,3-oxazolidin-3-yl)-2-fluorophenyl]piperazin-1-yl}-2-oxoethyl Acetate(11)

Step 1. Preparation of benzyl 4-[4-((5S)-5-{[(difluoroacetyl)amino]methyl}-2-oxo-1,3-oxazolidin-3-yl)-2-fluorophenyl]piperazine-1-carboxylate (8).

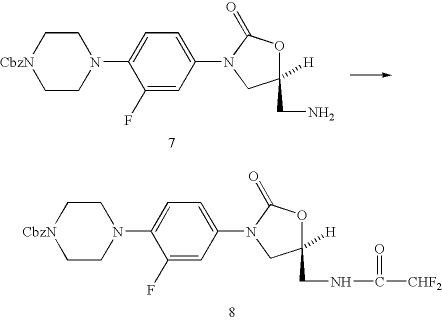

An ice cold stirred mixture of 7 (prepared according to the procedure described in U.S. Pat. No. 6,342,523, example 141) (2.00 g, 4.67 mmol), 1-(3-dimethylaminopropyl)-3-ethylcarbodiimide hydrochloride (EDC, 1.97 g, 10.3 mmol), 1-hydroxybenzotriazole hydrate (HOBT, 694 mg, 5.14 mmol), triethylamine (3.0 ml), difluoroacetic acid (0.38 ml, 6.07 mmol) and DMF (50 ml) is warmed to ambient temperature (24° C.), kept for 4 days and concentrated in vacuo. A mixture of the residue and CH₂Cl₂ is washed with water, dried (MgSO₄) and concentrated. Recrystallization of the residue from CH₂Cl₂-MeOH-hexane gave 1.47 g of the title compound (8). mp 147° C.; MS (EI) m/z 506 (M⁺).

Step 2. Preparation of 2,2-difluoro-N-{[(5S)-3-(3-fluoro-4-piperazin-1-ylphenyl)-2-oxo-1,3-oxazolidin-5-yl]methyl}acetamide (9).

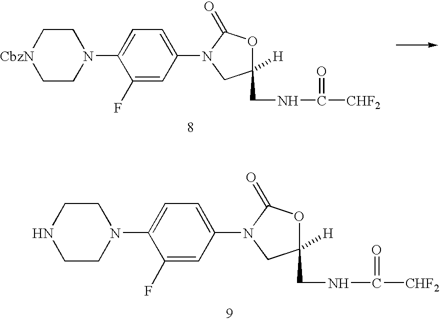

A mixture of 8 (1.40 g, 2.76 mmol), concentrated hydrochloric acid (0.690 mL, 8.28 mmol), 10% palladium-on-carbon catalyst (350 mg) and 95% EtOH (48 mL), is hydrogenated at an initial pressure of 40 psi for 18 hours and filtered through celite. The solid is washed with 40% H₂O-EtOH and the filtrate is concentrated to remove EtOH. The resulting solution is neutralized with saturated NaHCO₃ and extracted with 15% MeOH—CH₂Cl₂. The extract is dried (MgSO₄) and concentrated to give 892 mg of the title compound (9) as a white foam: MS (EI) m/z 372 (M⁺).

Step 3. Preparation of 2,2-difluoro-N-{[(5S)-3-(3-fluoro-4-piperazin-1-ylphenyl)-2-oxo-1,3-oxazolidin-5-yl]methyl}ethanethioamide (10).

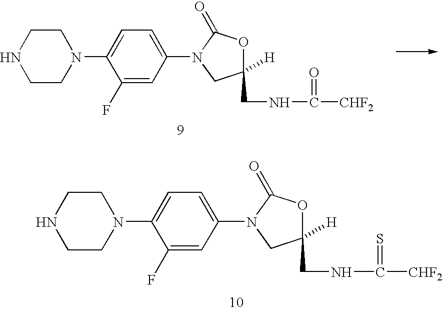

A stirred mixture of 9 (846 mg, 2.27 mmol), Lawesson's Reagent (1.84 g, 4.54 mmol), 1,3-dimethyl-3,4,5,6-tetrahydro-2(1H)-pyrimidinone (DMPU, 1.1 mL, 9.08 mmol) and THF (16 ml) is kept at ambient temperature (24° C.) for 18 hours and concentrated in vacuo. A mixture of the residue in 3N HCl is washed with CH₂Cl₂; the aqueous layer is then neutralized with solid NaHCO₃ and extracted with CH₂Cl₂. The extract is dried and concentrated to give 574 mg of the title compounds (10). MS (EI) m/z 388.1 (M⁺), 346.0, 344.1, 302.1, 208.1.

Step 4. Preparation of 2-{4-[4-((5S)-5-{[(2,2-Difluoroethanethiolyl)amino]methyl}-2-oxo-1,3-oxazolidin-3-yl)-2-fluorophenyl]piperazin-1-yl}-2-oxoethyl Acetate (11).

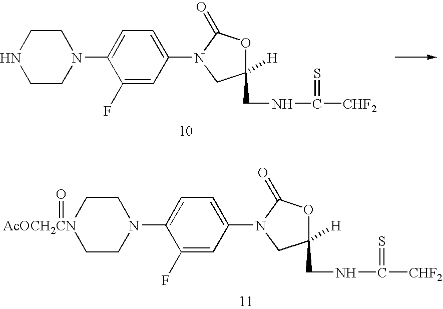

An ice cold stirred mixture of 10 (450 mg, 1.16 mmol), HOBT (172 mg, 1.28 mmol), acetoxyacetic acid (178 mg, 1.51 mmol) and DMF (9.2 ml) is treated with EDC (489 mg, 2.55 mmol), warmed slowly to ambient temperature (24° C.) and kept at this temperature for 2 days. It is then concentrated in vacuo and the residue is chromatographed on silica gel with 2-2.5% MeOH—CH₂Cl₂ to give 322 mg of the title compound (11) MS (EI) m/z 488.1 (M⁺), 444.1, 343.1, 308.1, 266.1.

### EXAMPLE 3

### Preparation of 2,2-difluoro-N-({(5S)-3-[3-fluoro-4-(4-glycoloyl piperazin-1-yl)phenyl]-2-oxo-1,3-oxazolidin-5-yl}methyl)ethanethioamide(12)

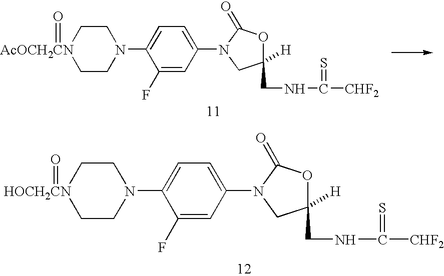

An ice cold, stirred solution of 11 (300 mg, 0.614 mmol) in MeOH (20 ml) is treated with 10% aqueous K₂CO₃ (1.4 ml), kept for 1 hour and treated with 1M KHSO₄ (5 ml) and water (20 ml). It is extracted with CH₂Cl₂ and the extract is dried (MgSO₄) and concentrated to give 280 mg of 12. A sample is chromatographed on silica gel with 5% MeOH—CH₂Cl₂ and the title compound (12) is crystallized from Et₂O-hexane and dried at 70° C. for 18 hours.

Physical data: MS (EI) m/z 446.0 (M⁺), 402.0, 266.1.

Anal calcd for C₁₈H₂₁F₃N₄O₄S; C, 48.43; H, 4.74; N, 12.55. Found: C, 48.19; H, 4.75; N, 12.47.

### Example 4

### Preparation of 2,2-difluoro-N-({(5S)-3-[4-glycoloylpiperazin-1-yl)phenyl]-2-oxo-1,3-oxazolidin-5-yl}methyl)ethanethioaamide(14)

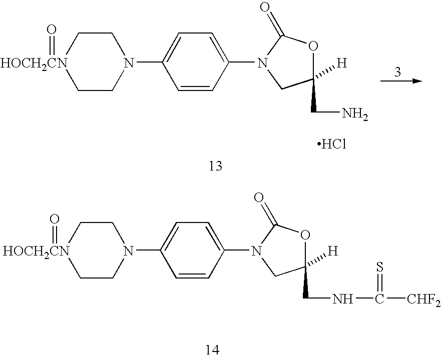

A stirred, ice cold suspension of 13 (prepared according to the procedure described in U.S. Pat. No. 6,342,523, Example 65) (504.4 mg, 1.36 mmol) in CH₂Cl₂ (5 ml), under nitrogen is treated with triethylamine (0.29 ml, 2.08 mmol) and then with a solution of 3 (489 mg, 1.60 mmol) in CH₂Cl₂ (1 ml). It is kept in the ice bath for 15 min and at ambient temperature (24° C.) for 30 min and then concentrated in vacuo. Chromatography of the residue on silica gel with 50% EtOAc-CH₂Cl₂ followed by mixtures of MeOH—CH₂Cl₂ containing 2.5-5% MeOH and crystallization of the product from CH₂Cl₂-EtOAc-hexane gave 357 mg of the title compound (14).

Physical data: mp 158-159° C.

HRMS calcd for C₁₈H₂₃F₂N₄O₄S (M+⁺) 429.1408, found 429.1411.

Anal. Calcd for C₁₈H₂₂F₂N₄O₄S: C, 50.46; H, 5.18; N, 13.08; S, 7.48. Found: C, 50.22; H, 5.19; N, 12.92; S, 7.40.

### Example 5

### Preparation of N-{[(5S)-3-(4-{4-[(benzyloxy)acetyl]piperazin-1-yl}phenyl)-2-oxo-1,3-oxazolidin-5-yl]methyl}-2,2-difluoroethanethioamide(16)

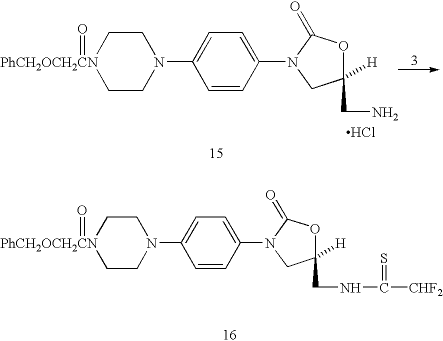

As described for the preparation of 14, the reaction of 15 ((prepared according to the procedure described in U.S. Pat. No. 6,342,523) with 3 and triethylamine in CH₂Cl₂ gave the title compound (16) which is purified by silica gel chromatography with 1-2% MeOH—CH₂Cl₂ and crystallization from acetone-hexane.

Physical data: mp 166-168° C.

MS (ESI+) m/z 519.1 (M+H⁺), 541.3 (M+Na⁺); MS (ESI−) m/z 517.1 (M−H).

HRMS calcd for C₂₅H₂₉F₂N₄O₄S (M+H⁺) 519.1877, found 519.1882.

Anal calcd for C₂₅H₂₈F₂N₄O₄S: C, 57.90; H, 5.44; N, 10.80. Found: C, 58.73; H, 5.71; N, 10.62.

### Example 6

### Preparation of (5S)-5-{[(2,2-difluoro-1-sulfinylethyl)amino]methyl}-3-[3-fluoro-4-(4-glycoloylpiperazin-1-yl)phenyl]-1,3-oxazolidin-2-one(17)

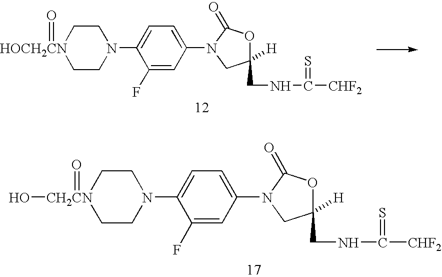

A stirred, ice cold mixture of 12 (0.42 g, 0.94 mmol) and MeOH (5 ml), under nitrogen, is treated, dropwise with a mixture of selenium dioxide (0.105 g, 0.946 mmol) and 30% hydrogen peroxide (0.12 ml) in water (1.5 ml), kept in the ice bath for 35 min and diluted with water (6 ml). It is extracted with CH₂Cl₂; the extract is washed with water and brine, dried (Na₂SO₄) and concentrated in vacuo without heating. Chromatography of the residue on silica gel with 50-100% acetone-CH₂Cl₂ and crystallization of the product from acetone gave 0.09 g of the title compound (18).

Physical data: mp 103-104° C.

HRMS calcd for C₁₈H₂₂F₃N₄O₅S (M+H⁺) 463.1263, found 463.1269.

Anal calcd for C₁₈H₂₁F₃N₄O₅S. (CH₃)₂CO: C, 48.46; H, 5.23; N, 10.76. Found: C, 48.55; H, 5.30; N, 10.73.

### Example 7

### Pyridinium 2-{4-[4-((5S)-5-{[(2,2-Difluoroethanethiolyl)amino]-methyl}-2-oxo-1,3-oxazolidin-3-yl)-2-fluorophenyl]piperazin-1-yl}-2-oxoethyl sulfate (18)

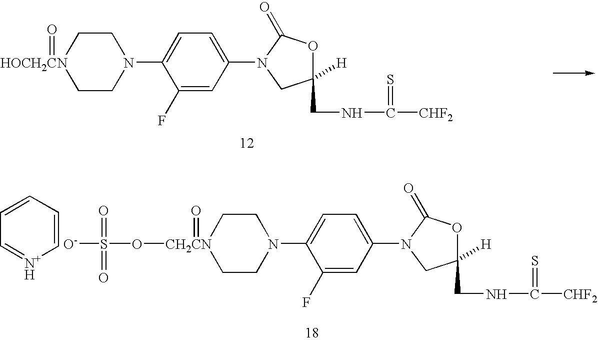

A stirred mixture of 12 (0.31 g, 0.695 mmol) and DMF (3 ml), under nitrogen, was treated, portionwise during 5 minutes, with pyridine-sulfur trioxide complex (0.384 g, 2.76 mmol) and the resulting solution was kept at ambient temperature for 50 min and then concentrated in vacuo. The residue was cooled in an ice bath and treated during 5 min with a solution of NaHCO₃ (0.23 g, 2.7 mmol) in water (7 ml). The resulting white solid was collected by filtration and dried in vacuo to give 0.36 g of 17: mp 205-206° C. (dec); MS (ESI−) m/z 524.95 (M−H); HRMS calcd for C₁₈H₂₀F₃N₄O₇S₂+H₂ 527.0881, found 527.0873; IR (DRIFT) 3233, 3300-2500 (broad) 1751, 1662, 1645 cm⁻¹. Anal. Calcd for C₂₃H₂₆F₃N₅O₇S₂: C, 45.61H, 4.33; N, 11.56, S, 10.59. Found: C, 44.79; H, 4.43; N, 11.44; S, 10.64.

### Example 8

### Preparation of 2,2-difluoro-N-({(5S)-3-[3-fluoro-4-(1-glycoloylpiperidin-4-yl)phenyl]-2-oxo-1,3-oxazolidin-5-yl}methyl)ethanethioamide (27). Step 1

Preparation of (5S)-5-(aminomethyl)-3-(3-fluoro-4-piperidin-4-ylphenyl)-1,3-oxazolidin-2-one (20).

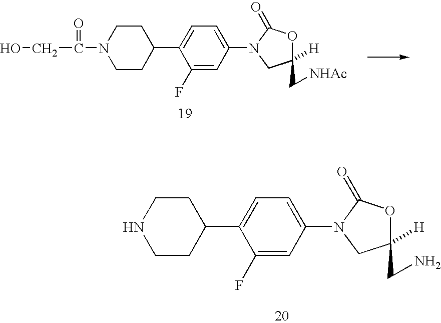

A stirred mixture of 19 (Prepared according to the procedure described in U.S. Pat. No. 6,342,523) (3.70 g, 9.40 mmol) in 6N HCl (94 ml) is warmed at 75-80° C. for 7 hours, cooled in an ice bath, adjusted to pH 13 with solid NaOH, saturated with NaCl and extracted with CH₂Cl₂. The extract is dried (Na₂SO₄) and concentrated in vacuo. Chromatography of the residue on silica gel with 5-15% MeOH-1% NH₄OHCH₂Cl₂ gave 1.78 g of the title compound (20).

Step 2. Preparation of (5S)-5-[(benzylideneamino)methyl]-3-(3-fluoro-4-piperidin-4-ylphenyl)-1,3-oxazolidin-2-one (21).

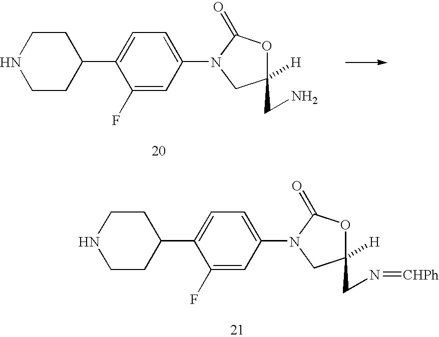

A stirred mixture of 63 (1.77 g, 6.03 mmol) and benzaldehyde (612 μL) in toluene (120 ml) is refluxed, under nitrogen for 5 hours, cooled to ambient temperature (24° C.) and treated with Na₂SO₄. It is stirred for 18 hours, filtered, and concentrated in vacuo to give the title compound (21).

Step 3. Preparation of (5S)-5-[(benzylideneamino)methyl]-3-(4-{1-[(benzyloxy)acetyl]piperidin-4-yl}-3-fluorophenyl)-1,3-oxazolidin-2-one (22).

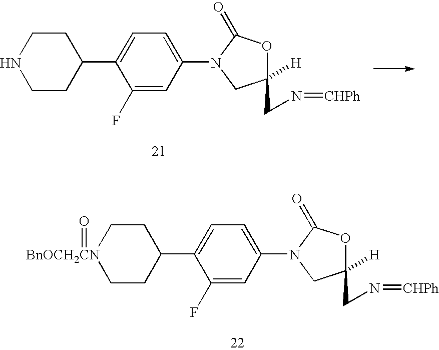

An ice cold, stirred solution of the product (21) from Step 2 in CH₂Cl₂ (30 ml) under nitrogen is treated with triethylanmine (1.26 ml, 9.04 mmol) and benzyloxyacetyl chloride (1.00 mL, 6.33 mmol), kept in the ice bath for 2 hours and diluted with CH₂Cl₂. It is washed with water and brine, dried (Na₂SO₄) and concentrated in vacuo to give the title compound (22). MS (ESI−) m/z 528 (M−H).

Step 4. Preparation of (5S)-5-(aminomethyl)-3-(4-{1-[(benzyloxy)acetyl]piperidin-4-yl}-3-fluorophenyl)-1,3-oxazolidin-2-one (23)

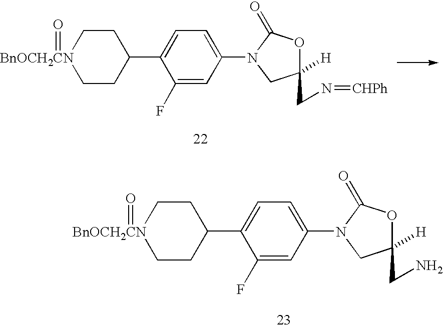

A mixture of the product (22) from Step 3, 10% palladium-on-carbon catalyst (1.28 g) and MeOH (60 ml) is hydrogenated at an initial pressure of 20-40 psi for 42 hours and filtered through celite. The filtrate is concentrated in vacuo and the residue is chromatographed on silica gel with 2.5-15% MeOH—CH₂Cl₂ to give the title compound (23). HRMS calcd for C₂₄H₂₉FN₃O₄ (M+H⁺) 442.2142, found 442.2144.

Step 5. Preparation of tert-butyl [(5S)-3-(4-{1-[(benzyloxy)acetyl]piperidin-4-yl}-3-fluorophenyl)-2-oxo-1,3-oxazolidin-5-yl]methylcarbamate (24).

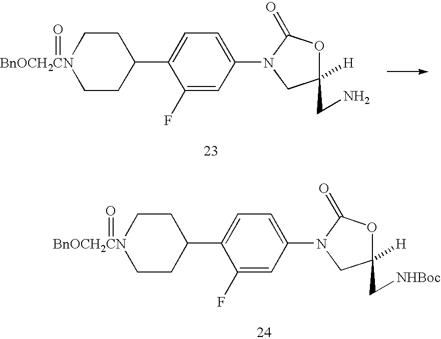

A stirred solution of 23 (340 mg, 0.793 mmol) in CH₂Cl₂ (8 ml), under nitrogen is treated with di-tert-butyl dicarbonate (190 mg, 0.872 mmol) and kept at ambient temperature for 2 hours. It is then diluted with CH₂Cl₂, washed with water, saturated NaHCO₃ and brine, dried (Na₂SO₄) and concentrated in vacuo. Chromatography of the residue on silica gel with 1-2% MeOH—CH₂Cl₂ gave the title compound (24). MS (ESI+) m/z 542 (M+H⁺), 564 (M+Na⁺).

Step 6. Preparation of tert-butyl {(5S)-3-[3-fluoro-4-(1-glycoloylpiperidin-4-yl)phenyl]-2-oxo-1,3-oxazolidin-5-yl}methylcarbamate (25).

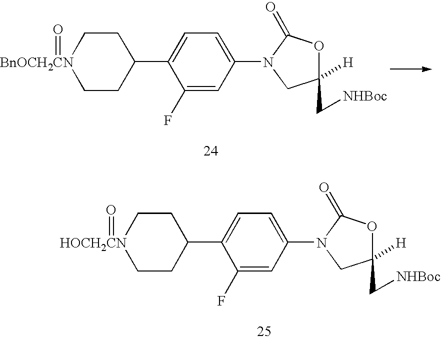

A mixture of 24 (365 mg, 0.674 mmol) and 10% palladium-on-carbon catalyst (144 mg) in MeOH (13 ml) is hydrogenated at an initial pressure of 40 psi for 2.5 hours and filtered through celite. The filtrate is concentrated in vacuo to give the title compound (25). MS (ESI+) m/z 452 (M+H⁺), 474 (M+Na⁺).

Step 7. Preparation of (5S)-5-(aminomethyl)-3-[3-fluoro-4-(1-glycoloylpiperidin-4-yl)phenyl]-1,3-oxazolidin-2-one hydrochloride (26).

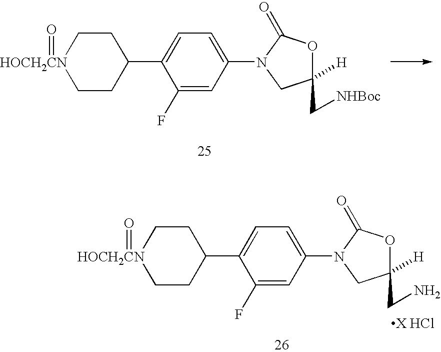

An ice cold, stirred solution of 25 (280 mg, 0.620 mmol) in MeOH (3 ml) is treated with 4M HCl in dioxane (6 ml), kept in the ice bath for 1 hour and concentrated in vacuo to give the title compound (26). MS (ESI+) m/z 352 (M+H⁺), MS (ESI−) m/z 386 (M+Cl).

Step 8. Preparation of 2,2-difluoro-N-({(5S)-3-[3-fluoro-4-(1-glycoloylpiperidin-4-yl)phenyl]-2-oxo-1,3-oxazolidin-5-yl}methyl)ethanethioamide(27).

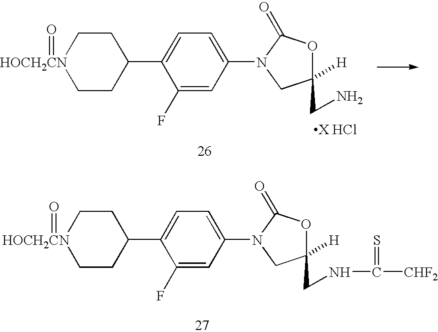

As described in the step 2, Example 1, a mixture of 26 and triethylamine in CH₂Cl₂ is allowed to react with 3 to give the title compound (27) which is purified by silica gel chromatography with 1-2% MeOH—CH₂Cl₂ and crystallization from EtOAc-hexane.

Physical data: mp 151-152° C. MS (ESI+) m/z 446 (M+H⁺), 468 (M+Na⁺).

MS (ESI−) m/z 444 (M−H). Anal calcd for C₁₉H₂₂F₃N₃O₄S: C, 51.23; H, 4.98; N, 9.43. Found: C, 51.23; H, 5.05; N, 9.35.

### Example 9

### Preparation of 2,2-difluoro-N-({(5S)-3-[4-(1-glycoloylpiperidine-4-yl)phenyl]-2-oxo-1,3-oxazolidin-5-yl}methyl)ethanethioamide(29)

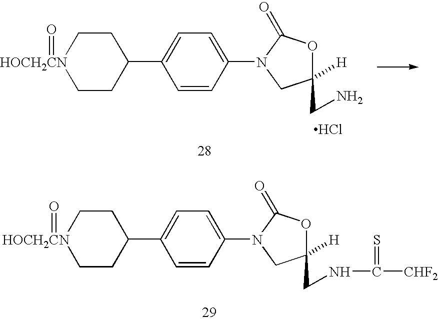

Following the procedure described in Example 7 but using a non-subsitituted phenyl starting material, compound 28 is prepared. Condensation of 28 with 3 and triethylamine in CH₂Cl₂ gave the title compound (29) which is purified by silica gel chromatography with 1-4% MeOH—CH₂Cl₂ and recrystallization from EtOAc-hexane.

Physical data: mp 140-141° C.

BRMS calcd for C₁₉H₂₄F₂N₃O₄S (M+H⁺) 428.1455, found 428.1426.

Anal. calcd for C₁₉H₂₃F₂N₃O₄S: C, 53.39; H, 5.42; N, 9.83. Found: C, 53.34; H, 5.40; N, 9.77.

Pharmaceutical Salts

The compound of formula I may be used in its native form or as a salt. In cases where forming a stable nontoxic salt is desired, administration of the compound as a pharmaceutically acceptable salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, ketoglutarate, and glycerophosphate. Suitable inorganic salts may also be formed, including hydrochloride, hydrobromide, sulfate, nitrate, bicarbonate, and carbonate salts. Pharmaceutically acceptable salts may be obtained using standard procedures well known in the art, for example by reacting a compound of the present invention with a suitable acid affording a physiologically acceptable anion.

Doses for Individual/Combination Therapy

In combating the infective diseases caused by gram-positive organisms, the compound of the formula I can be used either individually, or in combination with other antibiotics that are active against gram-positive organisms. Some of the gram-positive antibiotics may also have activity against gram-negative organisms.

Examples of such gram-positive antibiotics are listed in Table 1.

TABLE 1
Gram-Positive Antibiotics That May Be Used
In a Combination Therapy With The Compound of Formula I

| AGENTS | LO DOSE | | HI DOSE | | STD DOSE | |
|---|---|---|---|---|---|---|
| AMINOGLYCOSIDES | | | | | | |
| Amikacin | | | | | 15 | mg/kg/day |
| Gentamicin | 1 | mg/kg/day | 5 | mg/kg/day | | |
| | .5 | mg/kg | 2.5 | mg/kg | | |
| Spectinomycin | | | 40 | mg/kg | | |
| Tobramycin | 1 | mg/kg/day | 5 | mg/kg/day | | |
| | .5 | mg/kg/day | 5 | mg/kg/day | | |
| PENEMS | | | | | | |
| Imipenem/cilastatin | 62.5 | mg | 1 | g | | |
| | 6.25 | mg/kg | 25 | mg/kg | | |
| Meropenem | | | | | 40 | mg/kg |
| | .5 | mg/kg | 2.5 | mg/kg | | |
| 1^{ST} GEN CEPHS | | | | | | |
| Cefadroxil | .25 | g/day | 2 | g/day | | |
| | | | | | 30 | mg/kg/day |
| Cefazolin | 62.5 | mg | 1.5 | g | | |
| | 6.25 | mg/kg/day | 100 | mg/kg/day | | |
| Cephalexin | 62.5 | mg | 500 | mg | | |
| | 6.25 | mg/kg/day | 50 | mg/kg/day | | |
| 2^{ND} GEN CEPHS | | | | | | |
| Cefaclor | 62.5 | mg | 500 | mg | | |
| | 5 | mg/kg/day | 40 | mg/kg/day | | |
| Cefotetan | 0.125 | g | 3 | g | | |
| | 10 | mg/kg/day | 80 | mg/kg/day | | |
| Cefoxitin | .25 | g | 3 | g | | |
| | 20 | mg/kg/day | 160 | mg/kg/day | | |
| Cefprozil | 62.5 | mg | 500 | mg | | |
| | 1.87 | mg/kg/dose | 15 | mg/kg/dose | | |
| Cefuroxime | 187.5 | mg | 3 | g | | |
| | 31.25 | mg | 500 | mg | | |
| | 12.5 | mg/kg/day | 150 | mg/kg/day | | |
| | 31.25 | mg/kg/day | 500 | mg/kg/day | | |
| Loracarbef | 50 | mg | 400 | mg | | |
| | 3.75 | mg/kg/day | 500 | mg/kg/day | | |
| 3^{RD} GEN CEPHS | | | | | | |
| Cefdinir | 75 | mg | | | 600 | mg |
| Cefixime | 50 | mg | | | 400 | mg |
| Cefoperazone | .5 | g/day | 12 | g/day | | |
| | 25 | mg/kg/day | 150 | mg/kg/day | | |
| Cefotaxime | .25 | g | 2 | g | | |
| | 12.5 | mg/kg/dose | 300 | mg/kg/day | | |
| Cefpodoxime | 25 | mg | 400 | mg | 10 | mg/kg/day |
| Ceftazidime | 62.5 | mg | 2 | g q8 | | |
| | 25 | mg/kg/day | 150 | mg/kg/day | | |
| Ceftibuten | 2.25 | mg/kg | 400 | mg | 400 | mg |
| Ceftozoxime | .25 | g | 4 | g | | |
| | 12.5 | mg/kg/day | 200 | mg/kg/day | | |
| Ceftriaxone | 31.25 | mg | 2 | g | | |
| | 12.5 | mg/kg/day | 100 | mg/kg/day | | |
| 4^{TH} GEN CEPHS | | | | | | |
| Cefepime | 0.125 | g | 2 | g | | |
| | 12.5 | mg/kg | 50 | mg/kg q8 | | |
| MACROLIDES | | | | | | |
| Azithromycm | 62.5 | mg | 500 | mg | | |
| | 62.5 | mg | 500 | mg | | |
| Clarithromycin | 62.5 | mg | 500 | mg | 7.5 | mg/kg/day |
| Dirithromycin | | | | | 500 | mg |
| 1^{ST} GEN PENS | | | | | | |
| Penicillin G | 2 | million units/day | 30 | million units/day | | |
| | 2000 | units/kg/dy | 400,000 | units/kg/day | | |
| 2^{ND} GEN PENS | | | | | | |
| Cloxacillin | 62.5 | mg | 500 | mg | | |
| | 12.5 | mg/kg/day | 100 | mg/kg/day | | |
| Dicloxacillin | 31.25 | mg | 500 | mg | | |
| | 3.125 | mg/kg/day | 100 | mg/kg/day | | |
| Nafcillin | 125 | mg | 2 | g | | |
| | 2.5 | mg/kg | 25 | mg/kg | | |
| Oxacillin | 62.5 | mg | 2 | g | | |
| | 125 | mg | 1000 | mg | | |
| | 25 | mg/kg/day | 200 | mg/kg/day | | |
| | 12.5 | mg/kg/day | 100 | mg/kg/day | | |
| 3^{RD} GEN PENS | | | | | | |
| Amoxicillin | 62.5 | mg | 875 | mg | | |
| | 5 | mg/kg/day | 45 | mg/kg | | |
| Amoxicillin/clavulanic acid | 62.5 | mg | 875 | mg | | |
| | 6.25 | mg/kg/day | 45 | mg/kg/day | | |
| Ampicillin | 62.5 | mg | 12 | g/day q4 | | |
| | 6.25 | mg/kg/day | 300 | mg/kg/day | | |
| Ampicillin/sulbactam | 0.375 | g | 3 | g | 300 | mg/kg/day |
| 4^{TH} GEN PENS | | | | | | |
| Mezlocillin | 0.375 | g | 4 | g | 75 | mg/kg |
| Piperacillin | 1.5 | g/day | 24 | g day | | |
| | 25 | mg/kg/day | 300 | mg/kg/day | | |
| Piperacillin/tazobactam | | | 240 | mg/kg/day | | |
| Ticarcillin | .25 | g | 4 | g | | |
| | 12.5 | mg/kg/day | 300 | mg/kg/day | | |
| Ticarcillin/clavulanate | 50 | mg/kg/day | 300 | mg/kg/day | | |
| | 0.775 | g | 3.1 | g | | |
| 1^{ST} GEN QUINOLONES | | | | | | |
| Nalidixic Acid | | | 55 | mg/kg/day | | |
| 2^{ND} GEN QUINOLONES | | | | | | |
| Ciprofloxacin | 50 | mg | 750 | mg | | |
| | 2.5 | mg/kg/dose | 15 | mg/kg/dose | | |
| | 62.5 | mg | 750 | mg | | |
| | 2.5 | mg/kg/dose | 15 | mg/kg/dose | | |
| Enoxacin | 50 | mg | 400 | mg | | |
| Lomefloxacin | | | 400 | mg | | |
| Norfloxacin | | | 400 | mg | | |
| Ofloxacin | 50 | mg | 400 | mg | | |
| 3^{RD} GEN QUINOLONES | | | | | | |
| Levofloxacin | 62.5 | mg | 750 | mg | | |
| Sparfloxacin | 50 | mg | 400 | mg | | |
| 4^{TH} GEN QUINOLONES | | | | | | |
| Alatrofloxacin | 50 | mg | 300 | mg | | |
| Gatifloxacin | 50 | mg | 400 | mg | | |
| Moxifloxacin | | | 400 | mg | | |
| SULFAS | | | | | | |
| Trimethoprim/sulfamethoxazole | 15 | mg | 800 | mg | | |
| | 3.75 | mg/day | 150 | mg/day | | |
| Sulfisoxazole | 18.75 | mg | 150 | mg | | |
| Sulfamethoxazole | .25 | g | 2 | g | | |
| TETRACYCLINES | | | | | | |
| Doxycycline | 5 | mg | 100 | mg | | |
| Minocycline | 25 | mg | 200 | mg | | |
| Tetracycline | 62.5 | mg | 500 | mg | | |
| OTHER | | | | | | |
| Chloramphenicol | 12.5 | mg/kg/day | 100 | mg/kg/day | | |
| Clindamycin | 150 | mg | 900 | mg | | |
| | 37.5 | mg | 450 | mg | | |
| | 5 | mg/kg/day | 40 | mg/kg/day | | |
| | 2 | mg/kg/day | 25 | mg/kg/day | | |
| Quinupristin/dalfopristin | 1.875 | mg/kg | 7.5 | mg/kg q8 | | |
| Fosfomycin | | | 3 | g | | |
| Nitrofurantoin | 12.5 | mg | 100 | mg | | |
| | 1.25 | mg/kg/day | 7 | mg/kg/day | | |
| Rifampin | 2.5 | mg/kg | 600 | mg/kg | | |
| | 2.5 | mg/kg | 600 | mg/kg | | |
| Trimethoprim | 25 | mg | 200 | mg | 10 | mg/kg/day |
| Vancomycin | | | | | 1 | g |
| | 2.5 | mg/kg q6 | 15 | mg/kg q8 | | |

In combating the infective diseases caused by gram-positive and gram-negative organisms, the compound of the formula I can be used in combination with other antibiotics that are active against gram-negative organisms. Examples of such gram-negative antibiotics are listed in Table 2. Some of gram-negative antibiotics may also have activity against gram-positive organisms.

TABLE 2
Gram-Negative Antibiotics That May Be Used
In a Combination Therapy with The Compound of Formula I

| AGENTS | LO DOSE | | HI DOSE | | STD DOSE | |
|---|---|---|---|---|---|---|
| AMINOGLYCOSIDES | | | | | | |
| Amikacin | | | | | 15 | mg/kg/day |
| Gentamicin | 0.75 | mg/kg/day | 5 | mg/kg/day | | |
| | 0.5 | mg/kg | 2.5 | mg/kg | | |
| Spectinomycin | | | | | 40 | mg/kg |
| Tobramycin | 0.75 | mg/kg/day | 5 | mg/kg/day | | |
| | 0.5 | mg/kg/day | 5 | mg/kg/day | | |
| PENEMS | | | | | | |
| Imipenem/cilastatin | 62.5 | mg | 1 | g | | |
| | 6.25 | mg/kg | 25 | mg/kg | | |
| Merpenem | | | | | 40 | mg/kg |
| | 0.5 | mg/kg | 2.5 | mg/kg | | |
| 2^{ND} GEN CEPHS | | | | | | |
| Cefaclor | 62.5 | mg | 500 | mg | | |
| | 5 | mg/kg/day | 40 | mg/kg/day | | |
| Cefotetan | 0.125 | g | 3 | g | | |
| | 10 | mg/kg/day | 80 | mg/kg/day | | |
| Cefoxitin | 0.25 | g | 3 | g | | |
| | 20 | mg/kg/day | 160 | mg/kg/day | | |
| Cefprozil | 62.5 | mg | 500 | mg | | |
| | 1.875 | mg/kg/dose | 15 | mg/kg/dose | | |
| Cefuroxime | 187.5 | mg | 3 | g | | |
| | 31.25 | mg | 500 | mg | | |
| | 12.5 | mg/kg/day | 150 | mg/kg/day | | |
| | 31.25 | mg/kg/day | 500 | mg/kg/day | | |
| Loracarbef | 50 | mg | 400 | mg | | |
| | 3.75 | mg/kg/day | 500 | mg/kg/day | | |
| 3^{RD} GEN CEPHS | | | | | | |
| Cefdinir | 75 | mg | | | 600 | mg qd |
| Cefixime | 50 | mg | | | 400 | mg |
| Cefoperazone | 0.25 | g/day | 12 | g/day | | |
| | 25 | mg/kg/day | 150 | mg/kg/day | | |
| Cefotaxime | 0.25 | g | 2 | g | | |
| | 12.5 | mg/kg/dose | 300 | mg/kg/day | | |
| Cefpodoxime | 25 | mg | 400 | mg | 10 | mg/kg/day |
| Ceftazidime | 62.5 | mg | 2 | g q8 | | |
| | 25 | mg/kg/day | 150 | mg/kg/day | | |
| Ceftibuten | 2.25 | mg/kg | 400 | mg | 400 | mg |
| Ceftozoxime | 0.25 | g | 4 | g | | |
| | 12.5 | mg/kg/day | 200 | mg/kg/day | | |
| Ceftriaxone | 31.25 | mg | 2 | g | | |
| | 12.5 | mg/kg/day | 100 | mg/kg/day | | |
| 4^{TH} GEN CEPHS | | | | | | |
| Cefepime | 0.125 | g | 2 | g | | |
| | 12.5 | mg/kg | 50 | mg/kg q8 | | |
| MACROLIDES | | | | | | |
| Azithromycin | 62.5 | mg | 500 | mg | | |
| | 62.5 | mg | 500 | mg | | |
| Clarithromycin | 62.5 | mg | 500 | mg | 7.5 | mg/kg/day |
| Dirithromycin | | | | | 500 | mg |
| 3^{RD} GEN PENS | | | | | | |
| Amoxicillin | 62.5 | mg | 875 | mg | | |
| | 5 | mg/kg/day | 45 | mg/kg | | |
| Amoxicillin/clavulanic acid | 62.5 | mg | 875 | mg | | |
| | 6.25 | mg/kg/day | 45 | mg/kg/day | | |
| Ampicillin | 62.5 | mg | 12 | g/day q4 | | |
| | 6.25 | mg/kg/day | 300 | mg/kg/day | | |
| Ampicillin/sulbactam | 0.375 | g | 3 | g | 300 | mg/kg/day |
| 4^{TH} GEN PENS | | | | | | |
| Meziocillin | 0.375 | g | 4 | g | 75 | mg/kg |
| Piperacillin | 1.5 | g/day | 24 | g day | | |
| | 25 | mg/kg/day | 300 | mg/kg/day | | |
| Piperacillin/tazobactam | | | | | 240 | mg/kg/day |
| Ticarcillin | 0.25 | g | 4 | g | | |
| | 12.5 | mg/kg/day | 300 | mg/kg/day | | |
| Ticarcillin/clavulanate | 50 | mg/kg/day | 300 | mg/kg/day | | |
| | 0.775 | g | 3.1 | g | | |
| 1^{ST} GEN QUINOLONES | | | | | | |
| Nalidixic Acid | | | | | 55 | mg/kg/day |
| 2^{ND} GEN QUINOLONES | | | | | | |
| Ciprofloxacin | 50 | mg | 750 | mg | | |
| | 2.5 | mg/kg/dose | 15 | mg/kg/dose | | |
| | 62.5 | mg | 750 | mg | | |
| | 2.5 | mg/kg/dose | 15 | mg/kg/dose | | |
| Enoxacin | 50 | mg | 400 | mg | | |
| Lomefloxacin | | | | | 400 | mg |
| Norfloxacin | | | | | 400 | mg |
| Ofloxacin | 50 | mg | 400 | mg | | |
| 3^{RD} GEN QUINOLONES | | | | | | |
| Levofloxacin | 62.5 | mg | 750 | mg | | |
| Sparfloxacin | 50 | mg | 400 | mg | | |
| 4^{TH} GEN QUINOLONES | | | | | | |
| Alatrofloxacin | 50 | mg | 300 | mg | | |
| Gatifloxacin | 50 | mg | 400 | mg | | |
| Moxifloxacin | | | | | 400 | mg |
| SULFAS | | | | | | |
| Trimethoprim/sulfamethoxazole | 15/200 | mg | | | | |
| | 3.75 | mg/day | 150 | mg/day | | |
| Sulfisoxazole | 18.75 | mg | 150 | mg | | |
| Sulfamethoxazole | 0.25 | g | 2 | g | | |
| TETRACYCLINES | | | | | | |
| Doxycycline | 5 | mg | 100 | mg | | |
| Minocycline | 25 | mg | 200 | mg | | |
| Tetracycline | 62.5 | mg | 500 | mg | | |
| OTHER | | | | | | |
| Chloramphenicol | 12.5 | mg/kg/day | 100 | mg/kg/day | | |
| Aztreonam | 125 | mg | 2 | g | | |
| | 37.5 | mg | 450 | mg | | |
| | 5 | mg/kg/day | 40 | mg/kg/day | | |
| | 2 | mg/kg/day | 25 | mg/kg/day | | |
| Fosfomycin | | | | | 3 | g |
| Nitrofurantoin | 12.5 | mg | 100 | mg | | |
| | 1.25 | mg/kg/day | 7 | mg/kg/day | | |
| | 2.5 | mg/kg | 600 | mg/kg | | |
| Trimethoprim | 25 | mg | 200 | mg | 10 | mg/kg/day |

In Tables 1 and 2, the term “Lo Dose” means the recommended lower dosage for the combination therapy of the invention. It may be adjusted even lower depending on the requirements of each subject being treated and the severity of the bacterial infection. The lowest dosage possible may be 0.1 mg when combined with the compound of formula I of the present invention. The term “Hi Dose” means the recommended highest dosage in the combination therapy. It may be changed hereafter according to the US FDA standard. The term “Std Dose” means the recommended standard dosage for the combination therapy of the present invention. It may be adjusted even lower depending on the requirements of each subject being treated and the severity of the bacterial infection. A specific antibiotic may have more than one the recommended dosage ranges.

Generally, an antibacterially effective amount of dosage of the compound of formula I of the present invention, either administered individually or in combination with other antibiotics, will be in the range of about 0.1 to about 400 mg/kg of body weight/day, more preferably about 1.0 to about 50 mg/kg of body weight/day. It is to be understood that the dosages of active component(s) may vary depending upon the requirements of each subject being treated and the severity of the bacterial infection. In average, the effective amount of an active component is about 20 mg to 800 mg and preferable is about 200 mg to 600 mg per day.

The desired dose may conveniently be presented in a single dose or as divided into multiple doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation.

In cases of local administration or selective uptake, the effective local concentration of the drug may not be related to plasma concentration and other procedures know in the art may be used to determine the desired dosage amount.

For the combination therapy, the compound of formula I may be administered concurrently or concomitantly with other antibiotics. The term “concurrently” means the subject being treated takes one drug within about 5 minutes of taking the other drug. The term “concomitantly” means the subject being treated takes one drug within the same treatment period of taking the other drug. The same treatment period is preferably within twelve hours and up to forty-eight hours.

For the combination therapy, the compound of formula I, and one or more other antibiotics may be administered in the same physical form or separately, i.e., they may be administered in the same delivery vehicle or in different delivery vehicles.

For the combination therapy, some of the antibiotics may further be used with a β-Lactamase inhibitor. For example, Imipenem may be used with cilastatin, Ampicillin may be used with sulbactam, Piperacillin may be used with tazobactam, and Ampicillin may be used with sulbactam.

Specifically, the combination therapy of the present invention is the compound of formula I of the present invention with Amikacin.

Specifically, the combination therapy of the present invention is the compound of formula I of the present invention with Gentamicin.

Specifically, the combination therapy of the present invention is the compound of formula I of the present invention with Spectinomycin.

Specifically, the combination therapy of the present invention is the compound of formula I of the present invention with Tobramycin.

Specifically, the combination therapy of the present invention is the compound of formula I of the present invention with Imipenem/cilastatin.

Specifically, the combination therapy of the present invention is the compound of formula I of the present invention with Meropenem.

Specifically, the combination therapy of the present invention is the compound of formula I of the present invention with Cefadroxil.

Specifically, the combination therapy of the present invention is the compound of formula I of the present invention with Cefazolin.

Specifically, the combination therapy of the present invention is the compound of formula I of the present invention with Cephalexin.

Specifically, the combination therapy of the present invention is the compound of formula I of the present invention with Cefaclor.

Specifically, the combination therapy of the present invention is the compound of formula I of the present invention with Cefotetan.

Specifically, the combination therapy of the present invention is the compound of formula I of the present invention with Cefoxitin.

Specifically, the combination therapy of the present invention is the compound of formula I of the present invention with Cefprozil.

Specifically, the combination therapy of the present invention is the compound of formula I of the present invention with Cefuroxime.

Specifically, the combination therapy of the present invention is the compound of formula I of the present invention with Loracarbef

Specifically, the combination therapy of the present invention is the compound of formula I of the present invention with Cefdinir.

Specifically, the combination therapy of the present invention is the compound of formula I of the present invention with Cefixime.

Specifically, the combination therapy of the present invention is the compound of formula I of the present invention with Cefoperazone.

Specifically, the combination therapy of the present invention is the compound of formula I of the present invention with Cefotaxime.

Specifically, the combination therapy of the present invention is the compound of formula I of the present invention with Cefpodoxime.

Specifically, the combination therapy of the present invention is the compound of formula I of the present invention with Ceftazidime.

Specifically, the combination therapy of the present invention is the compound of formula I of the present invention with Ceftibuten.

Specifically, the combination therapy of the present invention is the compound of formula I of the present invention with Ceftozoxime.

Specifically, the combination therapy of the present invention is the compound of formula I of the present invention with Ceftriaxone.

Specifically, the combination therapy of the present invention is the compound of formula I of the present invention with Cefepime.

Specifically, the combination therapy of the present invention is the compound of formula I of the present invention with Azithromycin.

Specifically, the combination therapy of the present invention is the compound of formula I of the present invention with Clarithromycin.

Specifically, the combination therapy of the present invention is the compound of formula I of the present invention with Dirithromycin.

Specifically, the combination therapy of the present invention is the compound of formula I of the present invention with Penicillin G.

Specifically, the combination therapy of the present invention is the compound of formula I of the present invention with Cloxacillin.

Specifically, the combination therapy of the present invention is the compound of formula I of the present invention with Dicloxacillin.

Specifically, the combination therapy of the present invention is the compound of formula I of the present invention with Nafcillin.

Specifically, the combination therapy of the present invention is the compound of formula I of the present invention with Oxacillin.

Specifically, the combination therapy of the present invention is the compound of formula I of the present invention with Amoxicillin.

Specifically, the combination therapy of the present invention is the compound of formula I of the present invention with Amoxicillin/clavulanic acid.

Specifically, the combination therapy of the present invention is the compound of formula I of the present invention with Ampicillin.

Specifically, the combination therapy of the present invention is the compound of formula I of the present invention with Ampicillin/sulbactam.

Specifically, the combination therapy of the present invention is the compound of formula I of the present invention with Mezlocillin.

Specifically, the combination therapy of the present invention is the compound of formula I of the present invention with Piperacillin.

Specifically, the combination therapy of the present invention is the compound of formula I of the present invention with Piperacillin/tazobactam.

Specifically, the combination therapy of the present invention is the compound of formula I of the present invention with Ticarcillin.

Specifically, the combination therapy of the present invention is the compound of formula I of the present invention with Ticarcillin/clavulanate.

Specifically, the combination therapy of the present invention is the compound of formula I of the present invention with Nalidixic Acid.

Specifically, the combination therapy of the present invention is the compound of formula I of the present invention with Ciprofloxacin.

Specifically, the combination therapy of the present invention is the compound of formula I of the present invention with Enoxacin.

Specifically, the combination therapy of the present invention is the compound of formula I of the present invention with Lomefloxacin.

Specifically, the combination therapy of the present invention is the compound of formula I of the present invention with Norfloxacin.

Specifically, the combination therapy of the present invention is the compound of formula I of the present invention with Ofioxacin.

Specifically, the combination therapy of the present invention is the compound of formula I of the present invention with Levofloxacin.

Specifically, the combination therapy of the present invention is the compound of formula I of the present invention with Sparfloxacin.

Specifically, the combination therapy of the present invention is the compound of formula I of the present invention with Alatrofloxacin.

Specifically, the combination therapy of the present invention is the compound of formula I of the present invention with Gatifloxacin.

Specifically, the combination therapy of the present invention is the compound of formula I of the present invention with Moxifloxacin.

Specifically, the combination therapy of the present invention is the compound of formula I of the present invention with Trimethoprim/sulfamethoxazole.

Specifically, the combination therapy of the present invention is the compound of formula I of the present invention with Sulfisoxazole.

Specifically, the combination therapy of the present invention is the compound of formula I of the present invention with Sulfamethoxazole.

Specifically, the combination therapy of the present invention is the compound of formula I of the present invention with Doxycycline.

Specifically, the combination therapy of the present invention is the compound of formula I of the present invention with Minocycline.

Specifically, the combination therapy of the present invention is the compound of formula I of the present invention with Tetracycline.

Specifically, the combination therapy of the present invention is the compound of formula I of the present invention with Aztreonam.

Specifically, the combination therapy of the present invention is the compound of formula I of the present invention with Chloramphenicol.

Specifically, the combination therapy of the present invention is the compound of formula I of the present invention with Clindamycin.

Specifically, the combination therapy of the present-invention is the compound of formula I of the present invention with Quinupristin/dalfopristin.

Specifically, the combination therapy of the present invention is the compound of formula I of the present invention with Fosfomycin.

Specifically, the combination therapy of the present invention is the compound of formula I of the present invention with Metronidazole.

Specifically, the combination therapy of the present invention is the compound of formula I of the present invention with Nitrofurantoin.

Specifically, the combination therapy of the present invention is the compound of formula I of the present invention with Rifanpin.

Specifically, the combination therapy of the present invention is the compound of formula I of the present invention with Trimethoprim.

Specifically, the combination therapy of the present invention is the compound of formula I of the present invention with Vancomycin.

Routes of Administration

In therapeutic use for treating, or combating, bacterial infections in a mammal (i.e. human and animals) a compound of the present invention, its pharmaceutical compositions, or combining with other antibacterial agents can be administered orally, parenterally, topically, rectally, transmucosally, or intestinally.

Parenteral administrations include indirect injections to generate a systemic effect or direct injections to the afflicted area. Examples of parenteral administrations are subcutaneous, intravenous, intramuscular, intradermal, intrathecal, intraocular, intranasal, intravetricular injections or infuisions techniques.

Topical administrations include the treatment of infectious areas or organs readily accessibly by local application, such as, for example, eyes, ears including external and middle ear infections, vaginal, open wound, skins including the surface skin and the underneath dermal structures, or other lower intestinal tract. Topical administrations also include transdermal delivery to generate a systemic effect.

The rectal administration includes the form of suppositories.

The transmucosal administration includes nasal aerosol or inhalation applications.

The preferred routes of administration are oral and parenteral.

Corposition/Formulation

Pharmaceutical compositions of the present invention may be manufactured by processes well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, solutions, emulsions, gels, syrups, slurries, suspensions and the like, for oral ingestion by a patient. A carrier can be at least one substance which may also function as a diluent, flavoring agent, solubilizer, lubricant, suspending agent, binder, tablet disintegrating agent, and encapsulating agent. Examples of such carriers or excipients include, but are not limited to, magnesium carbonate, magnesium stearate, talc, sugar, lactose, sucrose, pectin, dextrin, mnnitol, sorbitol, starches, gelatin, cellulosic materials, low melting wax, cocoa butter or powder, polymers such as polyethylene glycols and other pharmaceutical acceptable materials.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identificatin or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, liquid polyethylene glycols, cremophor, capmul, medium or long chain mono-, fi- or triglycerides. Stabilizers may be added in these formulations, also.

Liquid form compositions include solutions, suspensions and emulsions. For example, there may be provided solutions of the compounds of this invention dissolved in water and water-propylene glycol and water-polyethylene glycol systems, optionally containing suitable conventional coloring agents, flavoring agents, stabilizers and thickening agents.

The compounds may also be formulated for parenteral administration, e.g., by injections, bolus injection or continuous infusion. Formulations for parenteral administration may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

For injection, the compounds of the invention may be formulated in aqueous solution, preferably in physiologically compatible buffers or physiological saline buffer. Suitable buffering agents include trisodium orthophosphate, sodium bicarbonate, sodium citrate, N-methylglucamine, L(+)-lysine and L(+)-arginine.

Parenteral administrations also include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use.

For suppository administration, the compounds may also be formulated by mixing the agent with a suitable non-irritating excipient which is solid at room temperature but liquid at rectal temperature and therefore will melt in the rectum to release the drug. Such materials include cocoa butter, beeswax and other glycerides.

For administration by inhalation, compounds of the present invention can be conveniently delivered through an aerosol spray in the form of solution, dry powder, or suspensions. The aerosol may use a pressurized pack or a nebulizer and a suitable propellant. In the case of a pressurized aerosol, the dosage unit may be controlled by providing a valve to deliver a metered amount. Capsules and cartridges of, for example, gelatin for use in an inhaler may be formulated containing a power base such as lactose or starch.

For topical applications, the pharmaceutical composition may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Aternatively, the pharmaceutical compositions can be formulated in a suitable lotion such as suspensions, emulsion, or cream containing the active components suspended or dissolved in one or more pharmaceutically acceptable carriers. Suitable carriers include, but are not limited to, mineral oil, sorbitan monostearate, polysorbate 60, cetyl esters wax, ceteary alcohol, 2-octyldodecanol, benzyl alcohol and water.

For ophthalmic and otitis uses, the pharmaceutical compositions may be formulated as micronized suspensions in isotonic, pH adjusted sterile saline, or preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

In addition to the formulations described previously, the compounds may also be formulated as depot preparations. Such long acting formulations may be in the forms of implants. A compound of this invention may be formulated for this route of administration with suitable biopolymers, hydrophbic materials, or as a sparing soluble derivative such as, without limitation, a sparingly soluble salt.

Additionally, the compounds may be delivered using a sustained-release system. Various sustained-release materials have been established and are well known by those skilled in the art. Sustained-release capsules may, depending on their chemical nature, release the compounds for 24 hours up to several days. Depending on the chemical natrue and the biological stability of the therapeutic reagent, additional strategies for protein stabilization may be employed.

The quantity of active component, that is the compound this invention, in the pharmaceutical composition and unit dosage form thereof may be varied or adjusted widely depending upon the manner of administration, the potency of the particular compound and the desired concentration. Determination of a therapeutically effective amount is well within the capability of those skilled in the art. Generally, the quantity of active component will range between 0.5% to 90% by weight of the composition.

We claim:

1. A compound of formula I 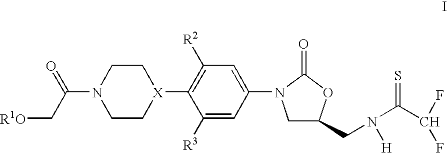
Or pharmaceutically acceptable salt
wherein X is N or CH;
R² and R³ are independently H or F;
R¹ is H, —CH₂phenyl, or —C(═O)C₁₋₄alkyl.

2. A compound of claim 1 wherein R² and R³ are H.

3. A compound of claim 1 wherein R² and R³ are F.

4. A compound of claim 1 wherein R² is H and R³ is F.

5. A compound of claim 2 wherein R¹ is H.

6. A compound of claim 3 wherein R¹ is H.

7. A compound of claim 4 wherein R¹ is H.

8. A compound of claim 5, 6, or 7 wherein X is N.

9. A compound of claim 5, 6, or 7 wherein X is CH.

10. A compound of claim 1 which is
(a) 2,2-Difluoro-N-({(5S)-3-[3,5-difluoro-4-(4-glycoloyl-piperazine-1-yl)phenyl]-2-oxo-1,3-oxazolidin-5-yl}methyl)ethanethioamide,
(b) 2-{4-[4-((5S)-5-{[(2,2-difluoroethanethiolyl)-amino]methyl}-2-oxo-1,3-oxazolidin-3-yl)-2-fluorophenyl]piperazin-1-yl}-2-oxoethyl Acetate,
(c) 2,2-difluoro-N-({(5S)-3-[3-fluoro-4-(4-glycoloylpiperazin-1-yl)phenyl]-2-oxo-1,3-oxazolidin-5-yl}methyl)ethanethioaamide,
(d) 2,2-difluoro-N-({(5S)-3-[4-glycoloylpiperazin-1-yl)phenyl]-2-oxo-1,3-oxazolidin-5-yl}methyl)-ethanethioamide, or
(e) N-{[(5S)-3-(4-{4-[(benzyloxy)acetyl]piperazin-1-yl}phenyl)-2-oxo-1,3-oxazolidin-5-yl]methyl}-2,2-difluoroethanethioamide.

11. A compound of claim 1 which is 2,2-difluoro-N-({(5S)-3-[3-fluoro-4-(4-glycoloylpiperazin-1-yl)phenyl]-2-oxo-1,3-oxazolidin-5-yl}methyl)ethanethioamide.

12. A compound of claim 1 which is
(a) 2,2-difluoro-N-({(5S)-3-[3-fluoro-4-(1-glycoloylpiperidin-4-yl)phenyl]-2-oxo-1,3-oxazolidin-5-yl}methyl)ethanethioamide, or
(b) 2,2-difluoro-N-({(5S)-3-[4-(1-glycoloylpiperidin-4-yl)phenyl]-2-oxo-1,3-oxazolidin-5-yl}methyl)ethanethioamide.

13. A compound of formula Ia useful as a intermediate for the preparation of a compound of formula I 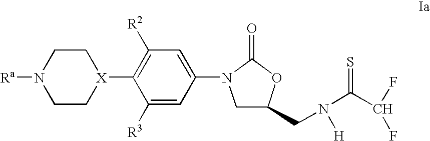
Wherein X is N or CH; R^{a} is H, —C(═O)OC(CH₃)₃, or —C(═O)OCH₂Ph; and R² and R³ are independently H or F.

14. A method for treating bacteria infections comprising administering to a mammal being treated a pharmaceutically effective amount of the compound of claim 1.

15. The method of claim 14 wherein the compound of claim 1 is administered parenterally, topically, rectally, or intranasally.

16. The method of claim 14 wherein the compound of claim 1 is administered orally.

17. The method of claim 15 wherein parenteral administration is subcutaneous, intravenous, intramuscular, intradermal, intrathecal, intraocular, intravetricular injection.

18. The method of claim 15 wherein said compound is administered in an amount of from about 0.1 to about 100 mg/kg of body weight/day.

19. The method of claim 15 wherein said compound is administered in an amount of from about 1 to about 50 mg/kg of body weight/day.

20. The method of claim 14 wherein said infection is skin infection.

21. The method of claim 14 wherein the infection is eye infection.

22. The method of claim 14 wherein the infection is ear infection.

23. The method of claim 14 wherein said mammal is human.

24. The method of claim 14 wherein said mammal is an animal.

25. A pharmaceutical composition comprising the compound of claim 1 or its pharmaceutically acceptable salts thereof and a pharmaceutically acceptable carrier.

26. A method for treating bacteria infections in a mammal comprising administering to said mammal (a) a pharmaceutically effective amount of the compound of claim 1 or a pharmaceutically effective salt thereof, and (b) a pharmaceutically effective amount of at least one antibiotic or a pharmaceutically effective salt thereof.

27. The method of claim 26 wherein the antibiotic is Amikacin, Gentamicin, Spectinomycin, Tobramycin, Imipenem, Meropenem, Cefadroxil, Cefazolin, Cephalexin, Cefaclor, Cefotetan, Cefoxitin, Cefprozil, Cefuroxime, Loracarbef, Cefdinir, Cefixime, Cefoperazone, Cefotaxime, Cefpodoxime, Ceftazidime, Ceftibuten, Ceftozoxime, Ceftriaxone, Cefepime, Azithromycin, Clarithromycin, Dirithromycin, Penicillin G, Cloxacillin, Dicloxacillin, Nafcillin, Oxacillin, Amoxicillin, Ampicillin, Mezlocillin, Piperacillin, Nalidixic Acid, Ciprofloxacin, Enoxacin, Lomefloxacin, Norfloxacin, Ofloxacin, Levofloxacin, Sparfloxacin, Alatrofloxacin, Gatifloxacin, Moxifloxacin, Trimethoprim, Sulfisoxazole, Sulfamethoxazole, Doxycycline, Minocycline, Tetracycline, Aztreonam, Chloramphenicol, Clindamycin, Quinupristin, Fosfomycin, Metronidazole, Nitrofurantoin, Rifampin, Trimethoprim, or Vancomycin.

28. A method for treating bacteria infections caused by gram-positive bacteria in a mammal comprising administering to said mammal (a) a pharmaceutically effective amount of compound of the formula I as shown in claim 1 or a pharmaceutically effective salt thereof, and (b) a pharmaceutically effective amount of at least one antibiotic or a pharmaceutically effective salt thereof.

29. The method of claim 28 wherein the antibiotic is Amikacin, Gentamicin, Spectinomycin, Tobramycin, Imipenem, Meropenem, Cefadroxil, Cefazolin, Cephalexin, Cefaclor, Cefotetan, Cefoxitin, Cefprozil, Cefuroxime, Loracarbef, Cefdinir, Cefixime, Cefoperazone, Cefotaxime, Cefpodoxime, Ceftazidime, Ceftibuten, Ceftozoxime, Ceftriaxone, Cefepime, Azithromycin, Clarithromycin, Dirithromycin, Penicillin G, Cloxacillin, Dicloxacillin, Nafcillin, Oxacillin, Amoxicillin, Ampicillin, Mezlocillin, Piperacillin, Nalidixic Acid, Ciprofloxacin, Enoxacin, Lomefloxacin, Norfloxacin, Ofloxacin, Levofloxacin, Sparfloxacin, Alatrofloxacin, Gatifloxacin, Moxifloxacin, Trimethoprim, Sulfisoxazole, Sulfamethoxazole, Doxycycline, Minocycline, Tetracycline, Chloramphenicol, Clindamycin, Quinupristin/dalfopristin, Fosfomycin, Nitrofurantoin, Rifampin, Trimethoprim, or Vancomycin.

30. A method for treating bacteria infections caused by gram-negative bacteria in a mammal comprising administering to said mammal (a) a pharmaceutically effective amount of the compound of claim 1 or a pharmaceutically effective salt thereof, and (b) a pharmaceutically effective amount of one or more antibiotics or a pharmaceutically effective salt thereof.

31. The method of claim 30 wherein the antibiotic is Amikacin, Gentamicin, Spectinomycin, Tobramycin, Imipenem, Meropenem, Cefaclor, Cefotetan, Cefoxitin, Cefprozil, Cefuroxime, Loracarbef, Cefdinir, Cefixime, Cefoperazone, Cefotaxime, Cefpodoxime, Ceftazidime, Ceftibuten, Ceftozoxime, Ceftriaxone, Cefepime, Azithromycin, Clarithromycin, Dirithromycin, Amoxicillin, Amoxicillin, Ampicillin, Ampicillin, Mezlocillin, Piperacillin, Piperacillin, Nalidixic Acid, Ciprofloxacin, Enoxacin, Lomefloxacin, Norfloxacin, Ofloxacin, Levofloxacin, Sparfloxacin, Alatrofloxacin, Gatifloxacin, Moxifloxacin, Trimethoprim, Sulfisoxazole, Sulfamethoxazole, Doxycycline, Minocycline, Tetracycline, Aztreonam, Chloramphenicol, Fosfomycin, Nitrofurantoin, or Trimethoprim.

32. The method of claims 26 wherein the compound of formula I and the other antibiotic are administered parenterally, topically, rectally, or intranasally.

33. The method of claims 26 wherein the compound of formula I and the other antibiotic are administered orally.

34. The method of claim 32 wherein parenteral administration is subcutaneous, intravenous, intramuscular, intradermal, intrathecal, intraocular, intravetricular injection.

35. The method of claims 26 wherein said infection is skin infection.

36. The method of claims 26 wherein said mammal is human.

37. The method of claims 26 wherein said mammal is an animal.

38. The method of claims 26 wherein the compound of formula I and the antibiotic are concomitantly administered.

39. The method of claims 26 wherein the compound of formula I and the antibiotics are concurrently administered.

40. A composition comprising:
(a) a pharmaceutically effective amount of the compound of formula I as shown in claim 1 or a pharmaceutically effective salt thereof, (b) a pharmaceutically effective amount of one or more antibiotics or a pharmaceutically effective salt thereof, and (c) a pharmaceutically acceptable carrier.

41. The composition of claim 40 wherein the antibiotic is Amikacin, Gentamicin, Spectinomycin, Tobramycin, Imipenem, Meropenem, Cefadroxil, Cefazolin, Cephalexin, Cefaclor, Cefotetan, Cefoxitin, Cefprozil, Cefuroxime, Loracarbef, Cefdinir, Cefixime, Cefoperazone, Cefotaxime, Cefpodoxime, Ceftazidime, Ceftibuten, Ceftozoxime, Ceftriaxone, Cefepime, Azithromycin, Clarithromycin, Dirithromycin, Penicillin G, Cloxacillin, Dicloxacillin, Nafcillin, Oxacillin, Amoxicillin, Ampicillin, Mezlocillin, Piperacillin, Nalidixic Acid, Ciprofloxacin, Enoxacin, Lomefloxacin, Norfloxacin, Ofloxacin, Levofloxacin, Sparfloxacin, Alatrofloxacin, Gatifloxacin, Moxifloxacin, Trimethoprirm, Sulfisoxazole, Sulfamethoxazole, Doxycycline, Minocycline, Tetracycline, Aztreonam, Chloramphenicol, Clindamycin, Quinupristin, Fosfomycin, Metronidazole, Nitrofurantoin, Rifampin, Trimethoprim, and Vancomycin.
